(19) United States
(12) Patent Application Publication
(10) Pub. No.: US 2005/0008623 A1
(43) Pub. Date: Jan. 13, 2005

(54) In vitro production of dendritic cells from CD14+ monocytes

(76) Inventors: Nicolas Bechetoille, Brignais (FR); Valerie Andre, Ampuis (FR); Colette Dezutter-Dambuyant, Vienne (FR); Isabelle Orly, Irigny (FR); Daniel Schmitt, Heyrieux (FR); Eric Perrier, Les Cotes D'Arey (FR)

Correspondence Address: MERCHANT & GOULD PC, P.O. BOX 2903, MINNEAPOLIS, MN, 55402-0903, US

(21) Appl. No.: 10/496,879

(22) PCT Filed: Dec. 10, 2002
(86) PCT No.: PCT/EP02/14874

(30) Foreign Application Priority Data
Dec. 10, 2001 (FR) 01/15942

(51) Int. Cl. A61K 48/00; C12N 5/08

(52) U.S. Cl. 424/93.21; 435/372

(57) ABSTRACT

The invention relates to the use of CD14⁺ monocytes for the production of dendritic cells.

The invention comprises the use of CD14⁺ monocytes isolated from peripheral circulating blood for obtaining, by differentiation, at least one mixed population of Langerhans cells and interstitial dendritic cells, both Langerhans cells and interstitial dendritic cells being preconditioned and undifferentiated, and/or differentiated and immature, and/or mature, and/or interdigitated. The invention comprises their use in suspension, monolayer and three-dimensional cell and tissue models. The invention comprises the use of these cells and of these models as study models for the assessment of immunotoxicity/immunotolerance, for the development of cosmetic and pharmaceutical active principles and for the development and implementation of methods of cell and tissue therapy.

The present invention relates essentially to a process for the in vitro culture of CD14⁺ monocytes, to a culture medium and to the use of the process in a method for the assessment of immunotoxicity/immunotolerance, in a method for the study and selection of active principles, in a method for the physio-pathological study of skin and mucous membranes and in a method of cell and/or tissue engineering and therapy.

### STATE OF THE ART

Dendritic cells (DC) are antigen-presenting cells which are considered to be guardians of the immune system. They are in fact located almost everywhere, namely in the thymus, the systemic circulation and the secondary lymphoid organs and also in the peripheral tissues such as the skin and mucous membranes, whether they can be monostratal or of the malpighian type, i.e. comprising a multistratal epithelium, namely those of the vagina, the outer cervix, the vulva, the perianal region, the esophagus and the mouth. Although in very small numbers in the organism, DC are at the center of the triggering of specific immune responses, exerting control over the specificity, intensity and nature of the immune response, and are located at the interface of innate and acquired immunity. Apart from their function of “switching on” the immune response, DC also have a role to play in the induction of peripheral tolerance.

DC precursors are derived from the differentiation of CD34⁺ hemopoietic precursors in the same way as numerous populations of the immune system and blood cells. They are transported by the blood to the skin and mucous membranes, where they differentiate and reside in the form of immature DC. Two types of DC can be described according to their in vivo location:
Langerhans cells (LC) are located in the malpighian-type epithelia (skin and mucous membranes) in greater or lesser density (from 100 to 1100/mm²). Their specific marker is Langerin (CD207), a protein involved in the formation of organelles observed on the electronic scale and named the Birbeck's granules. Apart from the markers Langerin and CD1a, LC express the antigens found on other DC at an immature stage, such as CD4, β₂-integrins and the adhesion molecules LFA-3 and ICAM-1. By virtue of their capacity to migrate towards the proximal lymph node after having captured an exoantigen while continuing their maturation, LC are responsible for numerous pathological conditions such as contact dermatitis and graft rejection reactions.
Interstitial dendritic cells (IDC) are found in the lamina propria of the mucous membranes and also in the dermis. In the latter case, they are also called dermal DC or dermal dendrocytes. These cells are devoid of Birbeck's granules and share numerous similarities and common markers with monocytes/macrophages. Furthermore, IDC express a specific marker, the lectin DC-SIGN, and have a similar allostimulant capacity to that of immature DC.

Following the capture of an antigen, LC and/or IDC migrate towards the lymph nodes. This migration correlates with an activation of the LC and/or IDC, with a modification of the expression of chemokine receptors (loss of expression of the CCR6 receptor and acquisition of expression of CCR7) and adhesion molecules, and with a modification of their phenotypic and functional characteristics. For example, in the case of LC, the Birbeck's granules become disorganized and their morphology is perturbed. In the lymphatic ganglia, the interaction between the CD40 receptor of the DC and its ligand CD40-L situated on the T lymphocytes induces a maturation of the DC into “interdigitated DC”, which are characterized by the membranous expression of the antigen CD83 and the co-stimulation markers CD80 and CD86, and by a massive membranous translocation of the class II molecules of the major histocompatibility complex, such as HLA-DR. These activated mature DC thus become producers of TNFα and IL-12.

A valuable use of LC, especially in combination with epithelial cells derived either from skin or from human mucous membranes, consists in integrating them into a system or model of “reconstructed skin” or “reconstructed mucous membrane” (cf. publication by Régnier, JID 1997; patent EP 0 789 074 to L'OREAL; Sivard P. Peaux et muqueuses reconstruites (Reconstructed skin and mucous membranes), Nouv. Dermatol., 2001, 20, 520-523). In particular, this could serve as a biological basis for methods said to be alternatives to animal experimentation, which should be increasingly used for in vitro evaluation of the tolerance and/or efficacy of products, such as pharmaceutical and cosmetic products.

In fact, these uses are currently limited, or even non-existent, due to the absence of a reasonably exploitable process for obtaining LC reliably on the industrial scale, and due to the imperfection of the models described.

Patent EP 0 789 074 to L'OREAL is concerned with a skin model or equivalent and the use of CD34⁺ precursors derived from umbilical cord blood. The skin equivalent is in fact only an epidermis equivalent since the cells are deposited on a matrix which is a de-epidermized dermis, i.e. a dead dermis containing no living cells.

Whatever the case may be, IDC are never obtained (nor are macrophages or endothelial cells) because the dermis is not “living”.

Furthermore, the number of CD34⁺ cells is limited since they are obtained from umbilical cord blood.

A publication by Geissmann (F. Geissmann, C. Prost, J-P. Monnet, M. Diy, N. Bruce and O. Hermine; 1998; J. Exp. Med., vol. 187, number 6, 961-966) describes the use of CD14⁺ monocytes obtained from circulating blood, as well as their culture in suspension for 6 days (in the presence of GM-CSF, TGFβ₁ and IL-4) to give LC.

According to the protocol described in said publication, the cells are cultivated in suspension and not on a three-dimensional model. Also, the presence of neither IDC nor other cells (macrophages, endothelial cells) is described.

### OBJECTS OF THE INVENTION

One main object of the present invention is to solve the novel technical problem consisting in the provision of a solution for the in vitro generation, from a single cellular precursor, of the two living populations of dendritic cells of the skin and the mucous membranes, namely Langerhans cells (or LC) and interstitial dendritic cells (or IDC).

Another main object of the present invention is to solve the novel technical problem consisting in the provision of a single precursor which is easily obtainable because it is present in the circulating blood and particularly in the peripheral circulating blood of a human or animal individual.

Another main object of the present invention is to solve the novel technical problem consisting in the provision of a single precursor which is present in sufficient quantity to allow the in vitro generation of cells in numbers such that they can be used on the industrial scale.

Another main object of the present invention is to solve the novel technical problem consisting in the provision of a single precursor which allows the in vitro generation of cells in a perfectly reproducible manner, particularly without variability as a function of the donor.

Another main object of the present invention is to solve the novel technical problem consisting in the provision of a single precursor which allows the rapid in vitro generation of cells (7 to 8 days of culture are required to obtain LC).

Another main object of the present invention is to solve the novel technical problem consisting in the provision of a single precursor which allows the in vitro generation of cells having the same phenotype and the same functions as those present in vivo.

Another main object of the present invention is to solve the novel technical problem consisting in the provision of a solution for the in vitro generation of dendritic cells, namely Langerhans cells and/or interstitial dendritic cells, at different, targeted steps of differentiation/maturation, i.e. at a step of preconditioned and undifferentiated cells, or at a step of differentiated and immature cells, or at a step of mature cells, or at a step of interdigitated cells.

Another main object of the present invention is to solve the novel technical problem consisting in the provision of a solution for the in vitro generation, from a single cellular precursor, of either predominantly Langerhans cells (or LC), or predominantly interstitial dendritic cells (or IDC), or a dual population of Langerhans cells and interstitial dendritic cells (or LC/IDC).

Another main object of the present invention is to solve the novel technical problem consisting in the provision of a solution for the in vitro generation, from a single cellular precursor of dendritic cells, namely Langerhans cells (or LC) and interstitial dendritic cells (or IDC), including the in vitro generation of subpopulations of these LC and/or CDI, these subpopulations being different ones from the others by their phenotypes and/or their functional properties.

Another object of the present invention is to solve the novel technical problem consisting in the provision of a solution for the use of these cells in therapy.

Another object of the present invention is to solve the novel technical problem consisting in the provision of a solution for the in vitro generation of dendritic cells, namely Langerhans cells and/or interstitial dendritic cells, for medical or biomedical applications such as anticancer cell therapy, for example an injection of DC capable of stimulating the immune response; cell therapy in cases of autoimmune disease through the creation of an immunotolerance situation, for example by producing anergic T cells; gene therapy for diseases affecting the immune system; and the development and production of vaccines.

Another main object of the invention is to solve the novel technical problem consisting in the provision of a solution for the in vitro generation of dendritic cells, namely Langerhans cells and/or interstitial dendritc cells, and for their integration into models, including models of skin tissues or mucous membranes.

Another main object of the present invention is to solve the novel technical problem consisting in the- provision of a solution for the in vitro generation of preconditioned cells which, when integrated into a complete skin or mucous membrane model, i.e. a model comprising both an epithelium and a connective matrix, are capable, by virtue of the cellular environment, preferably fibroblasts and epithelial cells, and the matricial environment, of locating in the epithelium in order to differentiate into Langerhans cells, and in the connective matrix in order to differentiate into interstitial dendritic cells, macrophages and endothelial cells, and of acquiring a functionality comparable to that of Langerhans cells, interstitial dendritic cells, macrophages and endothelial cells in vivo.

Another object of the present invention is to solve the novel technical problem consisting in the provision of a solution for the study and/or selection of substances, such as active principles.

Another object of the present invention is to solve the novel technical problem consisting in the provision of a solution for the in vitro generation of endothelial cells and macrophages.

Another object of the present invention is to solve the novel technical problem consisting in the provision of a solution for obtaining an equivalent of immunocompetent skin or mucous membrane.

Another object of the present invention is to solve the novel technical problem consisting in the provision of a model/tool for studying the physio-pathology of the different types of cells and tissues to which the invention relates, a model/tool for pharmacotoxicological study, for example with the aim of performing in vitro tests for predicting the immunotoxicity or allergenicity of external agents, and a model/tool for studying substances with immunomodulating properties.

Another object of the present invention is to solve the novel technical problem consisting in the provision of a solution for the use of these various models in therapy.

Another object of the present invention is to solve the novel technical problem consisting in the provision of a solution for the use of a model especially for the purpose of studying the immunostimulant or immunosuppressant activity of an active principle or evaluating or inducing an immunotolerance by said active principle.

Another object of the present invention is to solve the novel technical problem consisting in the provision of a solution for the use of a model for studying the physiopathology of epithelial barriers; irritation of skin or mucous membranes; aggressions of a biological nature, for example viruses, retroviruses such as HIV, bacteria, molds, microorganisms and particulate antigens; photo-toxicity; photoprotection; the effect of an active principle, particularly a cosmetic or pharmaceutical active principle; and the effect of finished products, particularly cosmetic or pharmaceutical products; and for studying the mechanisms of infection by a pathogenic agent.

Another object of the present invention is to solve the novel technical problem consisting in the provision of a solution for the use of a model for detecting the presence of a pathogenic agent, for example viruses, retroviruses such as HIV, bacteria, molds, microorganisms and particulate antigens.

Another object of the present invention is to solve the novel technical problem consisting in the provision of a solution for the use of a model for a medical, biomedical or cosmetic application, in particular for modulating the immune or tolerance response, in vitro or in vivo, following an environmental aggression, particularly of the physical type, such as UV irradiation, or of the chemical or biological type, particularly for the purpose of preventive or curative therapy.

Another object of the present invention is to solve the novel technical problem consisting in the provision of a solution for the use of a model for tissue and cell engineering applications; medical or biomedical applications such as anticancer cell therapy, for example an injection of DC capable of stimulating the immune response; cell therapy in cases of autoimmune disease through the creation of an immunotolerance situation, for example by producing anergic T cells; gene therapy for diseases affecting the immune system; and the development and production of vaccines.

### SUMMARY OF THE INVENTION

The present invention makes it possible for the first time to solve each of the above-mentioned technical problems in a safe, reliable and reproducible manner which can be used on the industrial and commercial scale and especially on the cosmetic and/or pharmaceutical and/or medical industrial scale.

The invention consists mainly in the in vitro generation, from a living single precursor, i.e. the CD14⁺ monocyte present in the peripheral circulating blood, of at least the two populations of dendritic cells of the skin and mucous membranes, namely Langerhans cells and interstitial dendritic cells.

Within the framework of the invention, the term “cells” is always to be understood as meaning “living cells”, unless indicated otherwise.

According to the invention, the term “peripheral circulating blood” is to be understood as meaning blood taken from any living being having a blood system in which the blood flows in a circuit, especially at the periphery, and particularly animals and mammals, preferably humans.

According to the invention, the term “fresh blood”, is blood from which the extraction of CD14⁺ monocytes is initiated and performed preferably not later than 24 hours after the taking of blood on an individual.

Thus, according to a first feature, the present invention relates to the use of CD14⁺ monocytes isolated from peripheral circulating blood for obtaining, by differentiation, at least one mixed population of Langerhans cells and of interstitial dendritic cells, both Langerhans cells and interstitial dendritic cells being preconditioned and undifferentiated, and/or differentiated and immature, and/or mature, and/or interdigitated.

According to one advantageous characteristic of the use of CD14⁺ monocytes, the extraction of CD14⁺ monocytes is performed from fresh blood i.e. initiated and performed preferably not later than 24 hours after taking of blood on an individual, preferably not later than 18 hours, preferably not later than 12 hours, preferably not later than 6 hours and still preferably the extraction is immediately initiated just after the taking of blood and performed not later than 5 hours.

According to one advantageous characteristic of the use of CD14⁺ monocytes, the differentiation results in the presence of different subpopulations of LC and/or IDC.

According to one advantageous characteristic of the use of CD14⁺ monocytes, the differentiation results in the presence of at least one additional subpopulation of preconditioned undifferentiated cells, and/or differentiated cells, such as cells of the macrophage type and/or cells of the endothelial type.

According to one advantageous characteristic of the use of CD14⁺ monocytes, the differentiation is effected by culture of these CD14⁺ monocytes in a culture medium containing at least the two cytokines GM-CSF and TGFβ, preferably TGFβ₁.

According to one advantageous characteristic of the use of these CD14⁺ monocytes, the distribution between the populations of LC and IDC depends on the presence of a third cytokine at a given concentration and for a given period of time during said culture, said cytokine preferably being the cytokine IL-13.

In another advantageous variant, the culture is carried out in the presence of the cytokine IL-13 for at most about two days so as to favor differentiation into LC, i.e. favor the predominant formation of LC.

In another advantageous variant, the culture is carried out in the presence of the cytokine IL-13 for about 6 days in order to favor the formation of IDC.

In another advantageous variant, the culture is carried out in the presence of the cytokine IL-13 for about 4 days in order to favor the formation of a dual population of LC/IDC.

According to another advantageous characteristic, an additional degree of differentiation of LC and IDC can be obtained by carrying out said culture in the presence of the cytokine TNFα.

The culture can advantageously be carried out in the presence of TNFα at a given concentration and for a given period of time, the latter being less than about 18 hours, in order to obtain immature Langerhans cells and immature interstitial dendritic cells while at the same time avoiding a maturation of these cells into mature activated dendritic cells.

According to another characteristic of the invention, the culture in the presence of TNFα is carried out at a given concentration and for a given period of time, the latter being more than about 20 hours, in order to obtain a maturation into mature activated dendritic cells.

According to another advantageous characteristic, the concentration of cytokine GM-CSF is between 0.1 and 4000 IU/ml, advantageously between 1 and 2000 IU/ml and more precisely about 400 IU/ml; the concentration of cytokine TGFβ, preferably TGFβ₁, is between 0.01 and 400 ng/ml, advantageously between 1 and 100 ng/ml and more precisely about 10 ng/ml; the concentration of cytokine IL-13, if this cytokine is present in the medium, is between 0.01 and 400 ng/ml, advantageously between 1 and 100 ng/ml and more precisely about 10 ng/ml; and the concentration of cytokine TNFα, if this cytokine is present in the medium, is between 0.1 and 4000 IU/ml, advantageously between 1 and 1000 IU/ml and more precisely about 200 IU/ml.

According to another advantageous characteristic of the use of the CD14⁺ monocytes, the LC and IDC obtained have functional phenotypes identical to those found in vivo.

According to another advantageous characteristic, the culture of said LC and IDC is carried out in a three-dimensional culture environment comprising, in particular, at least epithelial and stromal cells.

Advantageously, according to one characteristic of this additional differentiation, when the epithelial and stromal cells are distinctly separated, the LC are located mainly in the region of the epithelial cells and the IDC are located mainly in the region of the stromal cells.

Advantageously, according to one characteristic of the use of these CD14⁺ monocytes, endothelial cells and macrophages are obtained by differentiation from certain cells derived from the culture, particularly when they are placed in a three-dimensional environment.

Advantageously, according to one characteristic of the use, cells, preferably preconditioned cells, are obtained which, when integrated into a complete skin or mucous membrane model, i.e. a model comprising both an epithelium and a connective matrix, are capable, by virtue of the cellular environment, preferably fibroblasts and epithelial cells, and the matricial environment, of locating in the epithelium in order to differentiate into Langerhans cells, and in the connective matrix in order to differentiate into interstitial dendritic cells, macrophages and endothelial cells, and of acquiring a functionality comparable to that of Langerhans cells, interstitial dendritic cells, macrophages and endothelial cells in vivo.

According to a second feature, the present invention further relates to a process for the in vitro culture of CD14⁺ monocytes which comprises:
a) the separation, from circulating blood, of CD14⁺ monocytes previously harvested according to the state of the art, and
b) the culture of the separated CD14⁺ monocytes in a culture medium containing several cytokines for a sufficient period of time to obtain a dual population of LC and IDC.

According to one advantageous characteristic, in this process for the in vitro culture of CD14⁺ monocytes, the culture takes place in the presence of at least the cytokines GM-CSF and TGFβ, preferably TGFβ_{1 .}

According to another advantageous characteristic of the present invention, in the process for the in vitro culture of CD14⁺ monocytes, the culture takes place in the presence of a third cytokine at a given concentration and for a given period of time during said culture, said cytokine preferably being the cytokine IL-13.

In one variant of this advantageous characteristic, the culture is carried out in the presence of the cytokine IL-13 for at most about two days so as to favor differentiation into LC.

In another variant of this advantageous characteristic, the culture is carried out in the presence of the cytokine IL-13 for about six days in order to favor the formation of IDC.

In another advantageous variant of this characteristic, the culture is carried out in the presence of the cytokine IL-13 for about 4 days in order to favor the formation of a mixed population of LC/IDC.

According to one advantageous characteristic of the present invention, in the process for the in vitro culture of CD14⁺ monocytes, the culture takes place in the presence of the cytokine TNFα.

In one variant of this advantageous characteristic, the culture in the presence of TNFα is carried out at a given concentration and for a given period of time, the latter being less than about 18 hours, in order to obtain differentiation of the cells into still immature Langerhans cells and interstitial dendritic cells while at the same time avoiding a maturation into activated mature dendritic cells.

According to another advantageous characteristic, the culture in the presence of TNFα is carried out at a given concentration and for a given period of time, the latter being more than about 20 hours, in order to obtain a maturation into activated mature dendritic cells.

According to another advantageous characteristic of present invention, the extraction of CD14⁺ monocytes is performed from fresh blood i.e initiated and performed preferably not later than 24 hours after taking of blood on an individual, preferably not later than 18 hours, preferably not later than 12 hours, preferably not later than 6 hours and still preferably the extraction is immediately initiated just after the taking of blood and performed not later than 5 hours.

According to another advantageous characteristic of the present invention, in the process for the in vitro culture of CD14⁺ monocytes, the culture takes place in a three-dimensional culture environment, particularly in the presence of at least epithelial cells and stromal cells.

According to another advantageous characteristic of the present invention, an additional degree of differentiation is obtained by carrying out the culture of said Langerhans cells and interstitial dendritic cells in a three-dimensional culture environment comprising, in particular, at least distinctly separated epithelial and stromal cells.

According to another advantageous characteristic of the present invention, after culture with the cytokines in the process for the in vitro culture of CD14⁺ monocytes, a complementary stimulation of maturation is effected in particular by interaction of the dendritic cells with CD40-ligand, or by addition of the cytokine TNFα or lipopolysaccharide, for a sufficient period of time to obtain a phenotypic and functional maturation of said cells.

According to another advantageous characteristic of the present invention, the process for the in vitro culture of CD14⁺ monocytes comprises integration of a dual population of LC and IDC, in variable proportions, into a three-dimensional culture model.

In another variant of this last advantageous characteristic, the three-dimensional culture model includes skin models, mucous membrane models, dermis models, chorion models, epidermis models and epithelium models.

In another variant of this last advantageous characteristic, the three-dimensional culture model comprises a matricial support (of dermis or chorion) preferably selected from:
a collagen-based gel comprising stromal cells, particularly fibroblasts,
a porous matrix made of collagen which may contain one or more glycosaminoglycans and/or optionally chitosan (EP0296078A1 of the CNRS, WO 01/911821 and WO 01/92322 of COLETICA), these porous matrices possibly integrating stromal cells, particularly fibroblasts,
a gel or a membrane of hyaluronic acid (Hyalograft® 3D—Fidia Advanced Biopolymer) and/or of collagen and/or of fibronectin and/or fibrin (as, for example, Vitrix®—Organogenesis),
a dermal equivalent constitued of dermal layers (Michel M. et al ; 1999; In Vitro Cell. Dev Biol.-Animal ,vol. 35, 318-326),
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester (HATF) membrane, a semipermeable Biopore-CM membrane and a semipermeable polyester membrane, a polyglycolic acid membrane or film (this group contains products such as Skin²™ model ZK1100, Dermagraft® and Transcyte®—Advanced Tissue Science), said inert support possibly containing stromal cells, particularly fibroblasts.

In another variant of this last advantageous characteristic, the three-dimensional culture model used consists of the above-mentioned model onto whose surface epithelial cells, particularly keratinocytes, have been deposited.

In one variant of this last advantageous characteristic, the three-dimensional culture model used consists of a model into which has been incorporated at least one complementary cell type, for example nerve cells and/or endothelial cells (EC) and/or melanocytes and/or lymphocytes and/or adipocytes and/or appendages of skin, such as scalp hair, other body hair and sebaceous glands.

In another variant, certain cells derived from the culture differentiate into endothelial cells and macrophages, particularly when they are placed in a three-dimensional environment comprising at least epithelial and stromal cells.

The invention relates in general terms to a culture process comprising the use of CD14⁺ monocytes in a manner described above or in a manner resulting from the following description, including the Examples, taken in its entirety.

According to a third feature, the present invention relates to a medium for the in vitro culture of CD14⁺ monocytes which comprises a basic culture medium combined with at least two cytokines, namely the cytokine GM-CSF and the cytokine TGFβ, preferably TGFβ₁.

Advantageously, the culture medium combined with said two cytokines is also combined with the cytokine IL-13, which is preferably physically separated so that it can be introduced into the culture medium at a given moment during culture.

According to one advantageous characteristic of this third feature, the culture medium combined with said two cytokines is also combined with the cytokine TNFα, which is preferably physically separated so that it can be introduced into the culture medium at a given moment during culture.

According to another advantageous characteristic of this third feature, the concentration of cytokine GM-CSF in the culture medium is between 0.1 and 4000 IU/ml, advantageously between 1 and 2000 IU/ml and more precisely about 400 IU/ml; the concentration of cytokine TGFβ, preferably TGFβ₁, is between 0.01 and 400 ng/ml, advantageously between 1 and 100 ng/ml and more precisely about 10 ng/ml; the concentration of cytokine IL-13, if this cytokine is present in the medium, is between 0.01 and 400 ng/ml, advantageously between 1 and 100 ng/ml and more precisely about 10 ng/ml; and the concentration of cytokine TNFα, if this cytokine is present in the medium, is between 0.1 and 4000 IU/ml, advantageously between 1 and 1000 IU/ml and more precisely about 200 IU/ml.

According to a fourth feature, the invention relates to a cell population comprising at least one mixed population of Langerhans cells and interstitial dendritic cells—both Langerhans cells and interstitial dendritic cells being preconditioned and undifferentiated, and/or differentiated and immature, and/or mature, and/or interdigitated—which are obtainable from CD14⁺ monocytes and especially by the use as defined above, or by the culture process according to the above description, or by the use of the culture medium as described above.

According to a fifth feature, the invention relates to the use of the mixed population of LC and IDC obtained from the above-mentioned use of CD14⁺ monocytes, or by the above-mentioned culture process, or the use of the above-mentioned culture medium for the in vitro generation of dendritic cells, namely Langerhans cells and/or interstitial dendritic cells, for medical or biomedical applications such as anticancer cell therapy, for example an injection of DC capable of stimulating the immune response; cell therapy in cases of autoimmune disease through the creation of an immunotolerance situation, for example by producing anergic T cells; gene therapy for diseases affecting the immune system; and the development and production of vaccines.

Again, according to a sixth feature, the present invention relates to the use of the mixed population of LC and IDC obtained from the above-mentioned use of CD14⁺ monocytes, or by the above-mentioned culture process, or the use of the above-mentioned culture medium, or as described above, for the manufacture of a suspension, monolayer or three-dimensional, monocellular or multicellular study model.

According to one advantageous characteristic of this fifth feature, the study model is selected from:
a collagen-based gel comprising stromal cells, particularly fibroblasts,
a porous matrix made of collagen which may contain one or more glycosaminoglycans and/or optionally chitosan (EP0296078A1 of the CNRS, WO 01/911821 and WO 01/92322 of COLETICA), these porous matrices possibly integrating stromal cells, particularly fibroblasts,
a gel or a membrane of hyaluronic acid (Hyalograft® 3D—Fidia Advanced Biopolymer) and/or of collagen and/or of fibronectin and/or fibrin (as, for example, Vitrix®—Organogenesis),
a dermal equivalent constitued of dermal layers (Michel M. et al ; 1999; In Vitro Cell. Dev Biol.-Animal ,vol. 35, 318-326),
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester (HATF) membrane, a semipermeable Biopore-CM membrane and a semipermeable polyester membrane, a polyglycolic acid membrane or film (this group contains products such as Skin²™ model ZK1100, Dermagraft® and Transcyte®—Advanced Tissue Science), said inert support possibly containing stromal cells, particularly fibroblasts.

According to one advantageous characteristic, this model comprises mainly either LC, or IDC, or a mixture of LC/IDC, or a mixture of LC/IDC/endothelial cells/macrophages, or a mixture of IDC/endothelial cells/macrophages.

The tissue model is defined as being able to be an epidermis model consisting mainly of keratinocytes, a connective matrix model, called a dermis in the case of skin and chorion in the case of a mucous membrane, containing mainly stromal cells, an epithelium model consisting mainly of epithelial cells, a skin model consisting of an epidermis and a dermis, or a mucous membrane model consisting of an epithelium and a chorion.

Normal healthy cells, pathological cells or cells derived from lines can be used in these models; these cells can be of human or animal origin.

Epithelial cells, pigmentary cells, nerve cells etc. can be introduced into the epithelial part in addition to the cells generated according to the invention.

Stromal cells (particularly fibroblasts), T lymphocytes, adipocytes and appendages of skin (scalp hair, other body hair, sebaceous glands) can be introduced into the connective matrix in addition to the cells generated according to the invention.

According to a seventh feature, the present invention relates to a complete model of reconstructed skin or reconstructed mucous membrane, or a model of reconstructed dermis or reconstructed chorion, or a model of reconstructed epithelium, particularly an epidermis model, or any other suspension, monolayer or three-dimensional, monocellular or multicellular model comprising at least one mixed population of LC/IDC as obtained above from CD14⁺ monocytes.

According to one advantageous characteristic, this model of reconstructed tissue, or other model, is selected from:
a collagen-based gel comprising stromal cells, particularly fibroblasts,
a porous matrix made of collagen which may contain one or more glycosaminoglycans and/or optionally chitosan (EP0296078A1 of the CNRS, WO 01/911821 and WO 01/92322 of COLETICA), these porous matrices possibly integrating stromal cells, particularly fibroblasts,
a gel or a membrane of hyaluronic acid (Hyalograft® 3D—Fidia Advanced Biopolymer) and/or of collagen and/or of fibronectin and/or fibrin (as, for example, Vitrix®—Organogenesis),
a dermal equivalent constitued of dermal layers (Michel M. et al ; 1999; In Vitro Cell. Dev Biol.-Animal ,vol. 35, 318-326),
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester (HATF) membrane, a semipermeable Biopore-CM membrane and a semipermeable polyester membrane, a polyglycolic acid membrane or film (this group contains products such as Skin²™ model ZK1100, Dermagraft® and Transcyte®—Advanced Tissue Science), said inert support possibly containing stromal cells, particularly fibroblasts.

According to one advantageous characteristic, this model comprises mainly either LC, or IDC, or a mixture of LC/IDC, or a mixture of LC/IDC/endothelial cells/macrophages, or a mixture of IDC/endothelial cells/macrophages.

Advantageously, according to one characteristic of this model, the LC are located in the epithelial part and the IDC, macrophages and endothelial cells, when present, are located in the connective matrix.

Advantageously, the invention relates to a model as described above wherein cells are present which provide architecture, especially stromal cells, particularly fibroblasts, and/or epithelial cells, particularly keratinocytes, and/or other cell types, especially T lymphocytes, and/or nerve cells, and/or pigmentary cells, particularly melanocytes, and cells which provide immune defense, especially LC, IDC and/or macrophages, and cells which provide vascularization, especially endothelial cells, as well as adipocytes.

According to an eight feature, the present invention relates to the use of at least one of said mixed populations of LC and IDC as a model for the study and/or selection of active principles.

The term “active principle” is to be understood as meaning any substance, product or composition which is potentially capable of exhibiting an activity of value in industry, particularly in the cosmetic industry, pharmaceutical industry, dermopharmaceutical industry, food industry, agrifoodstuffs industry, etc.

An ninth feature of the invention relates to the use of an above-mentioned model especially for the purpose of studying the immunostimulant or immunosuppressant activity of an active principle or evaluating or inducing an immunotolerance by said active principle.

According to a tenth feature, the invention relates to the use of an above-mentioned model for studying the physiopathology of epithelial barriers; irritation of the skin or mucous membranes; aggressions of a biological nature, for example viruses, retroviruses such as HIV, bacteria, molds, microorganisms and particulate antigens; phototoxicity; photoprotection; the effect of active principles, particularly cosmetic or pharmaceutical active principles; and the effect of finished products, particularly cosmetic or pharmaceutical products; and for studying the mechanisms of infection by a pathogenic agent. In particular, the invention makes it possible to use the models for studying the mechanisms involved in the phenomena of infection, replication and transmission of viruses, including retroviruses such as HIV, and to research and develop therapeutic methods (including vaccines, drugs etc.).

According to a eleventh feature, the present invention relates to the use of an above-mentioned model for detecting the presence of a pathogenic agent, for example viruses, retroviruses such as HIV, bacteria, molds, microorganisms and particulate antigens.

According to an twelfth feature, the present invention relates to the use of an above-mentioned study model for a cosmetic, medical or biomedical application, in particular for modulating the immune or tolerance response, in vitro or in vivo, following an environmental aggression, particularly of the physical type, especially UV irradiation, or of the chemical or biological type, including the immunological type, particularly for the purpose of preventive or curative therapy.

According to a thirteenth feature of the present invention, the reconstructed tissue, reconstructed skin, reconstructed mucous membrane or study model can be used for tissue and cell engineering applications; medical or biomedical applications such as anticancer cell therapy, for example an injection of DC capable of stimulating the immune response; cell therapy in cases of autoimmune disease through the creation of an immunotolerance situation, for example by producing anergic T cells; gene therapy of diseases affecting the immune system; and the development and production of vaccines.

According to yet another feature, the present invention also covers any potentially active substance whose activity has been demonstrated through the use of at least the mixed population of cells obtained from CD14⁺ monocytes, especially by putting into effect any one of the foregoing features capable, in particular, of utilizing a study model.

By virtue of the invention, an easily accessible source of circulating monocytes is used through the possibility of using selectable donor blood bags. The number of CD14⁺ precursors present in circulating blood is high and makes it possible to produce a large number of LC and IDC in vitro with a high degree of reproducibility and feasibility.

In addition, the culture of CD14⁺ monocytes makes it possible to produce both LC and IDC, thereby providing a culture model suitable for the high-speed screening of substances intended in particular for applications to the skin or mucous membranes. This culture model therefore constitutes a satisfactory and complete tool because it utilizes at least LC and/or IDC at the same time; consequently, it constitutes an alternative method to animal experimentation and makes it possible especially to observe the ethical conventions in force according to the legislation of the cosmetic industry.

The invention also makes it possible to use the culture model in association with the models of reconstructed skin or reconstructed mucous membrane, affording the in vitro generation of a single model of “endothelialized immunocompetent reconstructed skin” or “endothelialized immunocompetent reconstructed mucous membrane” which is physiologically very similar to normal human skin or normal human mucous membrane. This model may be used for studying the physiopathology of epithelial barriers, irritation of the skin or mucous membranes, aggressions of a biological nature (for example viruses, retroviruses such as HIV, bacteria, molds, particulate antigens), phototoxicity, photoprotection, and the effect of active principles, particularly pharmaceutical and cosmetic active principles, and of finished products, particularly cosmetic and pharmaceutical products.

The invention makes it possible to generate different populations of DC whose different functionalities enable all the phenomena involved in the organism's infection/defense processes to be taken into account.

In addition, remarkably and unexpectedly, once integrated into a model of reconstructed skin or reconstructed mucous membrane, the cells generated in vitro from CD14⁺ monocytes, themselves isolated from peripheral circulating blood, are capable of:
locating in the epithelium in order to differentiate into LC;
locating in the connective matrix (dermis or chorion) in order to differentiate into IDC, endothelial cells and macrophages; and
acquiring a functionality comparable to that of LC, IDC, endothelial cells and macrophages in vivo.

It is seen that the invention affords major technical improvements allowing reliable and reproducible use on the industrial and commercial scale, particularly in the cosmetic and/or pharmaceutical industry, and that it can have major clinical implications.

A summary of the operating protocol used will give a better understanding of the different orientations of the CD14⁺ monocyte culture process.

Generation of Cells on the Basis of the Following Protocols, After Extraction of CD14⁺ Monocytes from Peripheral Circulating Blood:

Protocol 1:

CD14⁺ cultivated in suspension for 2 days in the presence of GM-CSF, TGFβ₁ and IL-13, then for an additional 4 days in the presence of GM-CSF and TGFβ₁→to D6: pre-LC (undifferentiated and immature)

Addition of TNFα (<18 h) in suspension→predominance of LC (differentiated and immature)

Protocol 2:

CD14⁺ cultivated in suspension for 6 days in the presence of GM-CSF, TGFβ₁ and IL-13→to D6: pre-IDC (undifferentiated and immature)

Addition of TNFα (<18 h) in suspension→predominance of IDC (differentiated and immature)

Protocol 3:

CD14⁺ cultivated in suspension for 4 days in the presence of GM-CSF, TGFβ₁ and IL-13, then for an additional 2 days in the presence of GM-CSF and TGFβ₁→to D6: pre-LC and pre-IDC (undifferentiated and immature)

Addition of TNFα (<18 h) in suspension→homogeneous mixed population of LC and IDC (differentiated and immature)

Protocol 4:

CD14⁺ cultivated in suspension for 6 days in the presence of GM-CSF, TGFβ₁ and IL-13, for either 2 days, 4 days or 6 days→to D6: pre-LC and pre-IDC (undifferentiated and immature)

Addition of TNFα (>20 h) in suspension→activated cells (differentiated and mature and no longer either LC or IDC)

If the cells obtained according to protocol 1, 2 or 3 are integrated into three-dimensional culture models (preferably at the undifferentiated cell stage=pre-LC and/or pre-IDC), it is observed that:

the addition of TNFα is not essential for differentiating the pre-LC and pre-IDC into LC and IDC; and
macrophages and dermal/chorionic endothelial cells are obtained spontaneously in addition to LC and IDC.
   Different Steps of Differentiation/Maturation of CD14⁺ Monocytes:
CD14⁺ monocyte→D6: pre-LC and/or pre-IDC (=undifferentiated and immature cells)
Addition of TNFα (<18 hours)→LC and/or IDC (=differentiated and immature cells)
Addition of TNFα (>20 hours)→mature cells which are no longer either LC or IDC (=activated mature cells)
Addition of CD40-ligand (present on the T lymphocytes) to the LC and/or IDC or to the mature cells→interdigitated cells (=last stage of maturation)

Other advantageous objects and characteristics of the invention will become clearly apparent to those skilled in the art from the following description referring to several Examples, which are given by way of illustration and cannot therefore in any way limit the scope of the invention.

In the Examples, the temperature is in degrees Celsius or is room temperature and the pressure is atmospheric pressure, unless indicated otherwise.

### EXAMPLE 1 OF THE INVENTION

Process for the Separation of CD14⁺ Monocytes from Peripheral Circulating Blood

Peripheral circulating blood is harvested by drawing venous blood from one or more human donors into vacutainers or plastic bags containing conventional anticoagulant products such as heparin-lithium or citrate phosphate dextran.

Advantageously, the CD14⁺ monocytes can be separated from this circulating blood according to the protocol described by Geissmann et al. in J. EXP. MED. vol. 187, no. 6, 16 March 1998, pages 961-966, published by The Rockefeller University Press, in the following manner:
After centrifugation on a Ficoll gradient, the mononuclear cells of the circulating blood are recovered and labeled indirectly with a cocktail of antibodies (mainly anti-CD3, anti-CD7, anti-CD19, anti-CD45RA, anti-CD56, anti-IgE) coupled with magnetic beads.
After passage over a magnetized column, only the monocytes which are not magnetically labeled are eluted.

The CD14⁺ monocytes are recovered from the eluate by any physical separation process well known to those skilled in the art, especially by sedimentation or centrifugation, and are eluted as such for the subsequent cultures.

Per 100 milliliters of blood withdrawn, about 150 million (±20 million) mononuclear cells are extracted and up to 40 million CD14⁺ monocytes are purified. Depending on the culture conditions used (cf. the Examples below), from 12 to 16 million Langerhans cells and/or interstitial dendritic cells are generated.

### EXAMPLE 2 OF THE INVENTION

Culture of Isolated CD14⁺ Monocytes to Give Undifferentiated and Immature Dendritic Cells

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 International Units/milliliter (or IU/ml) and the cytokine TGFβ1 at a rate of 10 nanograms/milliliter.

The culture is carried out at 37° C. in a humid atmosphere containing 5% of CO₂.

Within the framework of the invention, the culture medium is initially supplemented with a third cytokine, namely the cytokine IL-13 at a rate of 10 nanograms/milliliter. On day 4 of culture, the same culture medium devoid of IL-13 is added and the culture is continued for a further two days. On day 6 of culture, undifferentiated and immature dendritic cells are generated which are capable of orientating themselves towards the pathways of differentiation into Langerhans cells and interstitial dendritic cells:
about 30 to 50% of the dendritic cells generated in vitro express Langerin (specific marker of Langerhans cells) only at intracellular level and do not express the maturity markers CD83, DC-LAMP and CCR7;
about 30- to 50% of the dendritic cells generated in vitro express DC-SIGN (specific marker of interstitial dendritic cells) and do not express the maturity markers CD83, DC-LAMP and CCR7.

### EXAMPLE 3 OF THE INVENTION

Culture of Isolated CD14⁺ Monocytes to Give Undifferentiated and Immature Dendritic Cells Capable of Orientating Themselves Preferentially Towards the Pathway of Differentiation into Interstitial Dendritic Cells (IDC)

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ1 at a rate of 10 ng/ml.

The culture is carried out at 37° C. in a humid atmosphere containing 5% of CO₂.

Within the framework of the invention, the culture medium is initially supplemented with a third cytokine, namely the cytokine IL-13 at a rate of 10 ng/ml. After 6 days of culture, undifferentiated and immature dendritic cells are generated which are capable of orientating themselves preferentially towards the IDC differentiation pathway:
about 60 to 80% of the dendritic cells generated in vitro express DC-SIGN, which is the specific marker of interstitial dendritic cells;
the population of DC-SIGN+ cells is immature because the cells strongly express the marker CD68.

### EXAMPLE 4 OF THE INVENTION

Culture of Isolated CD14⁺ Monocytes to Give Undifferentiated and Immature Dendritic Cells Capable of Orientating Themselves Preferentially Towards the Pathway of Differentiation into Langerhans Cells (LC)

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 ng/ml.

The culture is carried out at 37° C. in a humid atmosphere containing 5% of CO₂.

The culture medium is initially supplemented with a third cytokine, namely the cytokine IL-13 at a rate of 10 ng/ml. Before 2 days of culture at the most, the same culture medium devoid of IL-13 is added up to day 6 of culture. On day 6, undifferentiated and immature dendritic cells are generated which are capable of orientating themselves preferentially towards the pathway of differentiation into Langerhans cells:
about 60 to 80% of the dendritic cells generated in vitro express Langerin at intracellular level and CCR6, which is the specific receptor of MIP-3α;
the dendritic cells generated in vitro are strongly chemoattracted by MIP-3a, demonstrating the functionality of the CCR6 receptor;
the dendritic cells generated in vitro are immature because they do not express the maturity markers CD83, DC-LAMP and CCR7.

### EXAMPLE 5 OF THE INVENTION

Culture of Isolated CD14⁺ Monocytes to Give Mainly Interstitial Dendritic Cells

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 ng/ml.

The culture is carried out at 37° C. in a humid atmosphere containing 5% of CO₂.

Within the framework of the invention, the culture medium is initially supplemented with a third cytokine, namely the cytokine IL-13 at a rate of 10 ng/ml. After 6 days of culture, the cytokine TNFα is added at a rate of 200 IU/ml over less than 18 hours to give mainly interstitial dendritic cells:
about 60 to 80% of the dendritic cells generated in vitro express DC-SIGN at membrane level;
the dendritic cells generated in vitro strongly express mannose receptors, a characteristic of interstitial dendritic cells;
the interstitial dendritic cells generated in vitro have the same functional characteristics as interstitial dendritic cells in vivo.

### EXAMPLE 6 OF THE INVENTION

Culture of Isolated CD14⁺ Monocytes to Give Mainly Langerhans Cells

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 ng/ml.

The culture is carried out at 37° C. in a humid atmosphere containing 5% of CO₂.

Within the framework of the invention, the culture medium is initially supplemented with a third cytokine, namely the cytokine IL-13 at a rate of 10 ng/ml. Before 2 days of culture at the most, the same culture medium devoid of IL-13 is added up to day 6 of culture. On day 6, the cytokine TNFα is added at a rate of 200 IU/ml over at most 18 hours to give mainly Langerhans cells:
about 60 to 80% of the dendritic cells generated in vitro express Langerin at membrane level (specific marker of Langerhans cells) and exhibit Birbeck's granules, which are ultrastructural specific markers of Langerhans cells;
the Langerhans cells generated in vitro have a similar functionality to that of Langerhans cells in vivo; they are capable of being chemoattracted by MIP-3α or of migrating under the effect of IL-1β or after sensitization by a potent allergen such as TNP or 2,4,6-trinitrobenzenesulfonic acid.

### EXAMPLE 7 OF THE INVENTION

Culture of Isolated CD14⁺ Monocytes to Give a Homogeneous Dual Population of Langerhans Cells and Interstitial Dendritic Cells

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ, at a rate of 10 ng/ml.

The culture is carried out at 37° C. in a humid atmosphere containing 5% of CO₂

Within the framework of the invention, the culture medium is initially supplemented with a third cytokine, namely the cytokine IL-13 at a rate of 10 ng/ml. After 4 days of culture, the same culture medium-devoid of IL-13 is added for a further 2 days. On day 6, the cytokine TNFα is added at a rate of 200 IU/ml over at most 18 hours, making it possible to generate a dual population of Langerhans cells and interstitial dendritic cells:
about 30 to 50% of the dendritic cells generated in vitro express Langerin at membrane level;
about 30 to 50% of the dendritic cells generated in vitro express DC-SIGN at membrane level;
dual labeling experiments confirm that the dendritic cells generated are either Langerin+ or DC-SIGN+.

### EXAMPLE 8 OF THE INVENTION

Culture of Isolated CD14⁺ Monocytes to Give Mainly Activated Mature Dendritic Cells

CD14⁺ monocytes, as obtained in Example 1, are cultivated at a rate of about 1 million per milliliter in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 ng/ml.

The culture is carried out at 37° C. in a humid atmosphere containing 5% of CO₂.

Within the framework of the invention, the culture medium is initially supplemented with a third cytokine, namely the cytokine IL-13 at a rate of 10 ng/ml. The culture is carried out up to day 6, irrespective of the incubation time of the cytokine IL-13. On day 6, the cytokine TNFα is added at a rate of 200 IU/ml over more than 20 hours to generate activated mature dendritic cells:
the dendritic cells generated in vitro express the maturation markers CD83, DC-LAMP and CCR7, which is the specific receptor of MIP-3β;
the dendritic cells generated in vitro are strongly chemoattracted by MIP-3β, demonstrating the functionality of the CCR7 receptor.

### EXAMPLE 9 OF THE INVENTION

Use of the Population of Mainly Langerhans Cells in a Suspension Monocellular Model of Migration

Generation of the cells: cf. Example 6.

To evaluate the migratory capacity of Langerhans cells generated in vitro towards any kind of aggression, for example an aggression of a biological nature, such as a microorganism, for example a microorganism of the bacterial type, migration chambers are used which have two compartments separated by a membrane with a porosity of 8 to 5 micrometers, which may or may not be covered with a matrix imitating a basal membrane (Matrigel™ type), or Boyden chamber, according to the following protocol:
2.5.10⁵ Langerhans cells generated in vitro are stimulated with 100 microliters of mannan at a concentration of 15 milligrams/milliliter for 10 minutes at 37° C.;
after this stimulation of bacterial type, the Langerhans cells are inoculated into the upper compartment of the migration chambers at a rate of 2.5.10⁵ cells in 0.5 milliliter of RPMI 1640 culture medium supplemented with 2% (v/v) of decomplemented fetal calf serum; 0.75 milliliter of RPMI 1640 culture medium supplemented with 2% of fetal calf serum has already been deposited in the lower compartment of the migration chambers;
after one hour of migration at 37° C., we recover the Langerhans cells which have migrated into the lower compartment of the migration chambers, namely the culture medium situated in the lower compartment of the migration chambers;
the Langerhans cells which have migrated are quantified by counting the cells under a white-light microscope;
the results are expressed as the migration index, i.e. the percentage of stimulated cells which have migrated, divided by the percentage of cells which have migrated spontaneously (negative control).

After simulation with mannan, the migration indices are between 1.6 and 1.9, i.e. the Langerhans cells generated in vitro and stimulated with mannan migrate 1.6 to 1.9 times more than the untreated Langerhans cells.

The Langerhans cells generated in vitro are capable of migrating under the effect of a stimulant, indicating that they are functional and that this test can be used as a study model for evaluating the effect of potentially aggressive/allergizing agents.

### EXAMPLE 10 OF THE INVENTION

Use of a Population of Mainly Interstitial Dendritic Cells in a Suspension Monocellular Model of Cytokine Secretion

Generation of the cells: cf. Example 5.

To quantify the protein secretion of cytokines secreted by interstitial dendritic cells generated in vitro, for example interleukin 12 or IL-12, towards any kind of aggression, for example an aggression of a chemical nature, particularly an allergen such as TNP or 2,4,6-trinitrobenzenesulfonic acid, we can use assays of the ELISA (Enzyme Linked Immuno-Sorbent Assay) type according to the following protocol:
1 million interstitial dendritic cells generated in vitro are stimulated with 800 microliters of TNP at a concentration of 5 millimolar for 10 minutes at 37° C.;
after this stimulation, the interstitial dendritic cells are recovered and inoculated into 6-well plates at a rate of 1 million cells/1 milliliter of RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 nanograms/milliliter;
after 48 hours of culture at 37° C. in a humid atmosphere containing 5% of CO₂, the culture supernatant of the interstitial dendritic cells is recovered;
the culture supernatants, which are first centrifuged at 1200 rpm for 10 minutes to remove the cellular debris, are used for ELISA; for the IL-12 ELISA procedure, reference may be made to the use instructions provided by the manufacturer (R&D System);
the results are expressed as the concentration of IL-12 in nanograms/1 million cells/milliliter.

The interstitial dendritic cells generated in vitro and stimulated with TNP secrete IL-12p75 at concentrations of between 2.1 and 2.7 nanograms IL-12p75/1 million cells/milliliter, whereas the untreated interstitial dendritic cells secrete IL-12p75 at concentrations of less than 0.1 nanogram/1 million cells/milliliter.

The interstitial dendritic cells generated in vitro increase their secretion of immunoactivating cytokine under the effect of a stimulant, indicating that they are functional and that this test can be used as a study model for evaluating the effect of potentially aggressive/allergizing agents.

### EXAMPLE 11 OF THE INVENTION

Use of a Dual Population of LC and IDC in a Suspension Bicellular Model of Antigen Internalization

Generation of the cells: cf. Example 7.

The advantage of a substantially homogeneous dual population of LC and IDC is the possibility of interacting with both cell types as in the in vivo situation. To study the internalization pathways of Langerhans cells and interstitial dendritic cells generated in vitro, i.e. their capacity to capture the antigen, we used dextran and the flow cytometry technique according to the following protocol:
2.10⁵ cells of a mixed population comprising Langerhans cells and interstitial dendritic cells generated in vitro are incubated successively with:
5 microliters of anti-DC-SIGN antibody at a concentration of 2 micrograms/milliliter for 30 minutes at 4° C.;
10 microliters of anti-mouse goat antibody coupled with the fluorochrome Tri-Color at a concentration of 1 microgram/milliliter for 30 minutes at 4° C.;
blocking with normal mouse serum diluted to 1/20;
2 microliters of anti-Langerin antibody coupled with phycoerythrin at a concentration of 1 microgram/milliliter for 30 minutes at 4° C.; and
1 milligram/milliliter of FITC-dextran in 500 microliters of internalization buffer made up of PBS (Phosphate Buffered Saline) supplemented with 1% of decomplemented fetal calf serum; the reaction is performed at 37° C. for the test and at 4° C. for the negative control for a period of 15 minutes;
after the reaction with FITC-dextran, the cells are analyzed by flow cytometry;
the results are expressed as the percentage of Langerhans cells and interstitial dendritic cells which are positive (compared with the negative control), i.e. as the percentage of cells which have internalized the dextran;
50 to 70% of the Langerhans cells (Langerin+) internalize FITC-dextran and 60 to 80% of the interstitial dendritc cells (DC-SIGN+) internalize FITC-dextran.

Dendritic cells generated in vitro are capable of internalizing antigens, indicating that they are functional and that this test can be used as a model for studying the internalization of antigens.

### EXAMPLE 12 OF THE INVENTION

Use of a Dual Population of LC and IDC in a Suspension Bicellular Model to Study Maturation Pathways of Both LC and IDC

Generation of the cells: cf. Example 7

On day 6, the cytokine TNFα is added at a rate of 200 IU/ml for 48 h.

The advantage of a substantially homogeneous dual population of LC and IDC is the possibility of interacting with both cell types as in the in vivo situation. To study the maturation pathways of LC and IDC generated in vitro, we analyzed the intracytoplasmic expression of maturation marker DC-LAMP on both LC and IDC. To this end, the experiments were performed according to the following protocol:
2.10⁵ cells of a mixed population comprising LC and IDC generated in vitro are incubated successively with:
either 5 microliters of anti-DC-SIGN antibody (2 micrograms/milliliter) or 10 microliters of anti-Langerin antibody (2 micrograms/milliliter), for 30 minutes at 4° C.;
10 microliters of anti-mouse goat antibody coupled with the fluorochrom FITC (Fluorescein IsoThioCyanate) at the concentration of 1 microgram/milliliter for 30 min at 4° C.;
blocking with normal mouse serum diluted to 1/20;
10 microliters of anti-DC-LAMP antibody coupled with fluorochrom PE (PhycoErythrin) at the concentration of 1 microgram/milliliter for 30 minutes at 4° C.
the results are expressed as the percentage of LC which are DC-LAMP positive and IDC which are DC-LAMP positive.
   After incubation of TNFα for 48 hours, the phenotype analyses reveal that IDC (DC-SIGN+) do not express maturation marker DC-LAMP whereas 50% of Langerin+LC population are DC-LAMP positive. These results outlined that the maturation process is distinguishable and consequently different in the two cutaneous DC subpopulations, i.e., LC and IDC. Then, active principles or ingredients (cosmetics or pharmaceuticals) which are capable to cross the skin barrier and to enter the superficial dermal compartment may provoke differential maturation pathways of both cutaneous DC, i.e. LC in epidermis and IDC in superficial dermis. Such approach may distinguish- potential tolerogenic from immunogenic active principles or ingredients (cosmetics or pharmaceuticals) in topical applications.

### EXAMPLE 13 OF THE INVENTION

Use of a Population of Activated Mature Dendritic Cells in a Suspension Monocellular Model of Allogenic Stimulation of Naive T Lymphocytes

Generation of the cells: cf. Example 8.

To study whether mature dendritic cells generated in vitro are capable of acquiring the functionality of interdigitated dendritic cells, i.e. capable of stimulating the proliferation of allogenic naive T lymphocytes, we performed mixed lymphocyte reactions according to the following protocol:
mature dendritic cells generated in vitro are cultivated at 37° C. in a humid atmosphere containing 5% of CO₂ for 48 hours with fibroblasts transfected with the molecule CD40-ligand, in a ratio of 10 activated dendritic cells to 1 fibroblast transfected with CD40-ligand, in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 nanograms/milliliter;
the activated dendritic cells are recovered and cultivated for 3 days with allogenic naive T lymphocytes in RPMI 1640 culture medium supplemented with 10% of human AB serum; a range of activated dendritic cells of between 125 and 8000 cells is prepared and cultivated with 10⁵ naive T lymphocytes;
on day 3 of the mixed lymphocyte culture, 20 microliters of tritiated thymidine with an activity of 5 millicuries are added over a period of 18 hours;
the results are expressed on a graph in which the number of activated dendritic cells (range from 125 to 8000 cells) is plotted on the abscissa and the incorporation of tritiated thymidine into the allogenic naive T lymphocytes, expressed in cpm or counts per minute, is plotted on the ordinate.

After interaction with CD40-ligand, the activated dendritic cells generated in vitro strongly stimulate the proliferation of naive T lymphocytes (between 12.10³ and 16.10³ cpm) compared with activated dendritic cells, which induce a low proliferation of naive T lymphocytes (between 3.10³ and 6.10³ cpm).

Dendritic cells generated in vitro are capable of acquiring the functionality of interdigitated dendritic cells, i.e. capable of acquiring high allostimulant capacities, indicating that they are functional -and that this test can be used as a model for studying allostimulation.

### EXAMPLE 14 OF THE INVENTION

Monolayer Multicellular Model of Keratinocytes and LC in Co-Culture

Generation of the cells: cf. Example 4 or 6.

1.10⁵ keratinocytes are inoculated into culture dishes of the 6-well plate type in a Clonetics medium (reference: KGM-2) for a period of immersion culture up to confluence of the keratinocytes. At the point of confluence, 1 to 3.10⁵ dendritic cells generated in vitro according to Example 4 or 6 are added. The culture is maintained for a further 3 to 4 days in RPMI 1640 culture medium supplemented with 10% of decomplemented fetal calf serum and initially containing two cytokines, namely the cytokine GM-CSF at a rate of 400 IU/ml and the cytokine TGFβ₁ at a rate of 10 nanograms/milliliter.

### EXAMPLE 15 OF THE INVENTION

Monolayer Multicellular Model of Fibroblasts and Interstitial Dendritic Cells in Co-Culture

Generation of the cells: cf. Examples 3 and 5.

1.10⁵ fibroblasts are inoculated into culture dishes of the 6-well plate type in DMEM-Glutamax medium supplemented with 10% of Hyclone II calf serum, penicillin at a concentration of 100 IU/milliliter and gentamicin at a final concentration of 20 micrograms/milliliter for a period of immersion culture up to confluence of the fibroblasts. At the point of confluence, 1 to 3.105 dendritic cells generated in vitro according to Example 3 or 5 are added. The culture is maintained for a further 3 to 4 days.

### EXAMPLE 16 OF THE INVENTION

Three-Dimensional Multicellular Model of Reconstructed Epidermis or Reconstructed Epithelium of Gingival Mucous Membrane Containing Epithelial Cells and Langerhans Cells

The model is prepared according to the following protocol:
1 to 2.10⁶ keratinocytes or epithelial cells are inoculated into inserts of the Boyden chamber type (membrane of porosity 0.4 μm); after one day of culture, 1 to 3.10⁵ dendritic cells generated in vitro according to Example 4 are added and the culture is continued in DMEM-Glutamax/Ham F-12 culture medium (ratio 3/1 v/v) supplemented with 10% of Hyclone II calf serum, ascorbic acid 2-phosphate at a final concentration of 1 millimolar, EGF at a final concentration of 10 ng/ml, hydrocorbsone at a final concentration of 0.4 microgram/milliliter, umuline at a final concentration of 0.12 IU/milliliter, isuprel at a final concentration of 0.4 microgram/ milliliter, triiodothyronine at a final concentration of 2.10⁻⁹ molar, adenine at a final concentration of 24.3 micrograms/milliliter, penicillin at a final concentration of 100 IU/milliliter, amphotericin B at a final concentration of 1 microgram/milliliter and gentamicin at a final concentration of 20 micrograms/milliliter, for a period of immersion culture of 3 to 8 days;
the keratinocyte cultures are then placed at the air-liquid interface for 12 to 18 days in the same culture medium as that used for the immersion culture, except for the calf serum, hydrocortisone, isuprel, triiodothyronine and umuline;
the epithelial cell cultures are then maintained as immersion cultures for 12 to 18 days in the same culture medium as that used for the immersion culture, except that the percentage of calf serum is reduced from 10% to 1%.

### EXAMPLE 17 OF THE INVENTION

Three-Dimensional Multicellular Models of Reconstructed Dermis or Reconstructed Gingival Chorion Containing Populations of Interstitial Dendritic Cells, Macrophages and Endothelial Cells

Generation of the cells: cf. Example 3, 4, 5, 6 or 7.

The model is prepared according to the following protocol:
2.10⁵ normal human fibroblasts of skin or gingival mucous membrane are inoculated onto a matricial substrate based on collagen crosslinked with diphenyl-phosphorylazide in DMEM-Glutamax culture medium supplemented with 10% of Hyclone II calf serum, ascorbic acid 2-phosphate at a final concentration of 1 millimolar, EGF or epidermal growth factor at a final concentration of 10 nanograms/milliliter, penicillin at a final concentration of 100 IU/milliliter, amphotericin B at a final concentration of 1 microgram/milliliter and gentamicin at a final concentration of 20 micrograms/milliliter. After 14 days of culture, 1 to 3.10⁵ dendritic cells generated in vitro are inoculated onto the connective matrix equivalent, which is cultivated for a further 14 days.

The markers used reveal the presence of interstitial dendritic cells (DC-SIGN+), macrophages (macrophage marker from Novocastra: clone 3A5-monoclonal antibody NCL-MACRO) and endothelial cells (V-CAM+).

### EXAMPLE 18 OF THE INVENTION

Three-Dimensional Multicellular Model of Reconstructed Skin Containing Populations of Langerhans Cells, Interstitial Dendritic Cells, Macrophaaes and Endothelial Cells

Generation of the cells: cf. Example 4 or 6.

The model is prepared according to the following protocol:
2.10⁵ normal human skin fibroblasts are inoculated onto a dermal substrate based on collagen/glycosaminoglycan/chitosan in DMEM-Glutamax culture medium supplemented with 10% of Hyclone II calf serum, ascorbic acid 2-phosphate at a final concentration of 1 millimolar, EGF or epidermal growth factor at a final concentration of 10 ng/ml, penicillin at a final concentration of 100 IU/milliliter, amphotericin B at a final concentration of 1 microgram/milliliter and gentamicin at a final concentration of 20 micrograms/milliliter, for a culture period of 14 days;
2.10⁵ normal human keratinocytes and 1 to 3.10⁵ dendritic cells generated in vitro are then inoculated onto the dermis equivalent in DMEM-Glutamax/Ham F-12 culture medium (ratio 3/1 v/v) supplemented with 10% of Hyclone II calf serum, ascorbic acid 2-phosphate at a final concentration of 1 millimolar, EGF at a final concentration of 10 ng/ml, hydrocortisone at a final concentration of 0.4 microgram/milliliter, umuline at a final concentration of 0.12 IU/milliliter, isuprel at a final concentration of 0.4 microgram/milliliter, triiodothyronine at a final concentration of 2.10⁻⁹ molar, adenine at a final concentration of 24.3 micrograms/milliliter, penicillin at a final concentration of 100 IU/milliliter, amphotericin B at a final concentration of 1 microgram/milliliter and gentamicin at a final concentration of 20 micrograms/milliliter, for a period of immersion culture of 7 days;
the cultures are then placed at the air-liquid interface for 14 days in the same culture medium as that used for the immersion culture, except for the calf serum, hydrocortisone, isuprel, triiodothyronine and umuline;
the cultures are then coated in an amorphous resin such as Tissue-Teck® and frozen in liquid nitrogen;
immunohistochemical studies are carried out on 6 micrometer thick, frozen sections in order to characterize the different cell types present using monoclonal antibodies directed against Langerin, DC-SIGN, V-CAM and macrophage marker from Novocastra: clone 3A5-monoclonal antibody NCL-MACRO;
the markers used reveal the presence of Langerhans cells (Langerin+) in the epidermis and interstitial dendritic cells (DC-SIGN+), macrophages (macrophage marker from Novocastra: clone 3A5-monoclonal antibody NCL-MACRO) and endothelial cells (V-CAM+) in the dermis.

### EXAMPLE 19 OF THE INVENTION

Three-Dimensional Multicellular Model of Pigmented Reconstructed Skin Containing Populations of Langerhans Cells, Interstitial Dendritic Cells, Macrophages and Endothelial Cells

The model is prepared according to the protocol described in Example 18, 10,000 melanocytes being co-inoculated with the keratinocytes and the dendritic cells generated in vitro.

In addition to the markers described in Example 18, the melanocytes are immunolabeled (MELAN-A) and an immunohistochemical study is carried out (DOPA reaction).

### EXAMPLE 20 OF THE INVENTION

Three-Dimensional Multicellular Model of Reconstructed Skin Containing Populations of Interstitial Dendritic Cells, Macrophages and Endothelial Cells

Generation of the cells: cf. Example 3, 5 or 7.

The model is prepared by following the protocol described in Example 18.

The markers used reveal the presence of interstitial dendritic cells (DC-SIGN+), macrophages (macrophage marker from Novocastra: clone 3A5-monoclonal antibody NCL-MACRO) and endothelial cells (V-CAM+) in the dermis.

### EXAMPLE 21 OF THE INVENTION

Three-Dimensional Multicellular Model of Reconstructed Vaginal Mucous Membrane Containing Populations of Langerhans Cells, Interstitial Dendritic Cells, Macrophacies and Endothelial Cells

Generation of the cells: cf. Example 4 or 6.

The model is prepared according to the protocol described in Example 18, with the following modifications: the keratinocytes are replaced with vaginal epithelial cells, the fibroblasts are derived from vaginal mucous membrane and the culture is carried out totally as an immersion culture in the culture medium.

The epithelial cell cultures are then maintained as immersion cultures for 12 to 18 days in the same culture medium, except that the percentage of calf serum is reduced from 10 to 1%.

The markers used reveal the presence of Langerhans cells (Langerin+) in the epithelium and interstitial dendritic cells (DC-SIGN+), macrophages (macrophage marker from Novocastra: clone 3A5-monoclonal antibody NCL-MACRO) and endothelial cells (V-CAM+) in the chorion.

### EXAMPLE 22 OF THE INVENTION

Three-Dimensional Multicellular Model of Reconstructed Vaginal Mucous Membrane Containing Populations of Interstitial Dendritic Cells, Macrophages and Endothelial Cells

Generation of the cells: cf. Example 3, 5 or 7.

The model is prepared according to the protocol described in Example 18, with the following modifications: the keratinocytes are replaced with vaginal epithelial cells, the fibroblasts are derived from vaginal mucous membrane and the culture is carried out totally as an immersion culture in the culture medium. The epithelial cell cultures are then maintained as immersion cultures for 12 to 18 days in the same culture medium as that used for the immersion culture, except that the percentage of calf serum is reduced from 10% to 1%.

The markers used reveal the presence of interstitial dendritic cells (DC-SIGN+), macrophages (macrophage marker from Novocastra: clone 3A5-monoclonal antibody NCL-MACRO) and endothelial cells (V-CAM+) in the chorion.

### EXAMPLE 23 OF THE INVENTION

Use of Any One of the Models Described in Example 16, 18, 19 or 20 for Studying LC/Epithelial Environment Interactions

After preparation of the model, the E-cadherin is labeled.

Expression of the adhesion molecule E-cadherin is found on the Langerhans cells and the epithelial cells, representing possible interactions of the heterophilic type via this protein between the Langerhans cells and the neighboring epithelial cells.

### EXAMPLE 24 OF THE INVENTION

Use of the Model of Reconstructed Epidermis Described in Example 16 for Studying the Influence of UVB Radiation

To study the influence of various environmental factors, particularly UV radiation and more precisely UVB, which mainly penetrates the epidermis, we evaluated the migration and the phenotypic profile of Langerhans cells in a model of reconstructed epidermis after UVB irradiation by means of immunohistochemical studies according to the following protocol:
after 11 days of culture, the reconstructed epidermides are irradiated with a dose of 0.5 joule/cm² of UVB and the cultures are continued for 3 days;
immunohistochemical studies for visualizing an epidermal depletion of the Langerhans cells are carried out using the anti-Langerin monoclonal antibody;
the phenotypic modifications of the Langerhans cells still present in the epidermal compartment of the reconstructed epidermides are detected using the monoclonal antibodies anti-CD1a, anti-CCR6, anti-HLA-DR, anti-CD80, anti-CD83, anti-CD86, anti-CCR7 and anti-DC-LAMP.

After UVB irradiation, an estimated decrease of more than 50% is observed in the number of Langerhans cells in the epidermal compartment, together with a decrease e.g. in the labeling intensity of the co-stimulation molecule CD86 on the Langerhans cells remaining in the epidermis.

### EXAMPLE 25 OF THE INVENTION

Use of the Model of Reconstructed Skin Described in Example 20 for Studying the Influence of UVA Radiation

To study the influence of various environmental factors affecting the dermis of the skin, particularly UV radiation and more precisely UVA, we evaluated the phenotypic profile of interstitial dendritic cells in a model of reconstructed skin after UVA irradiation by means of immunohistochemical studies according to the following protocol:
after 32 days of culture, the reconstructed skin samples are irradiated with a dose of 10 joules/cm² of UVA and the cultures are continued for a further 3 days;
immunohistochemical studies make it possible to detect phenotypic modifications of the interstitial dendritic cells present in the dermal compartment of the reconstructed skin samples by using the monoclonal antibodies anti-DC-SIGN, anti-clotting factor XIIIa, anti-HLA-DR, anti-CD80, anti-CD83, anti-CD86, anti-CCR7 and anti-DC-LAMP.

After UVA irradiation, a decrease is observed for example in the labeling intensity of the molecules HLA-DR and CD86 on the interstitial dendritic cells present in the dermis.

### EXAMPLE 26 OF THE INVENTION

Use of the Model of Reconstructed Skin Described in Any One of Examples 18, 19 and 20 for Studying the Profile of Cytokines Secreted Under the Effect of an Active Principle

To evaluate the potentially sensitizing or allergizing power and assess a possible pro- or anti-inflammatory activity of an active principle intended for the human skin, we quantified the secretion of pro-inflammatory cytokines such as IL-1, IL-6, IL-8, IL-12, TNFα, INFγ etc. and immunosuppressant cytokines such as IL-2, IL-10 etc. by ELISA according to the following protocol:
after 32 days of culture, retinol 10S is added to the culture medium at a final concentration of 0.05% over 7 days;
the cytokines are assayed every 2 to 3 days for 14 days.

It is observed that the retinol 10S causes a stimulation of the pro-inflammatory cytokines.

### EXAMPLE 26 OF THE INVENTION

Use of the Model of Reconstructed Epidermis Described in Example 16 for Screening Active Principles Capable of Modulating Allergic Reactions

The immunomodulating effect of an active principle after the induction of an allergizing stress is studied according to the following protocol:
on day 12 of culture, 300 microliters of TNP (2,4,6-trinitrobenzenesulfonic acid) are added at a concentration of 5 millimolar over 30 minutes at 37° C. to the upper compartment of the Boyden chamber;
after this stimulation, the culture medium is replaced with fresh medium possibly containing the active principles to be tested, at different concentrations, and the culture is continued for a further 2 days;
after 14 days of culture, the number of Langerhans cells which have migrated into the lower compartment of the Boyden chamber (membrane porosity from 8 to 5 μm, membrane not covered or covered with MATRIGEL™) is quantified by counting under the optical microscope; the culture medium is recovered and centrifuged and the supernatant is used for ELISA of the IL-12 (R&D System) and for assay of the proteins (BCA); the results are expressed as the concentration of IL-12 in nanograms/microgram of proteins.

The combined results of the migration test and the IL-12 synthesis make it possible to establish the immunomodulating profile of the active principles tested.

### EXAMPLE 28OF THE INVENTION

Use of a Model of Reconstructed Skin or Reconstructed Mucous Membrane Obtained According to Any One of Examples 18, 19 and 21 for Studying the Immunostimulant or Immunosuppressant Activity of an Active Principle or Evaluating and/or Inducing an Immunotolerance

To evaluate the capacity of Langerhans cells and/or interstitial dendritic cells to induce or not to induce immune and/or tolerogenic responses towards an active principle, we studied the phenotypic profile of Langerhans cells and/or interstitial dendritic cells by immunohistochemistry in the three-dimensional culture models according to the following protocol:
on day 32 of culture, the active principle is added to the culture medium at different concentrations and the culture is continued for 7 days;
the phenotypic profile of the cells is analyzed by immunohistochemistry with a series of antibodies (cf. Examples 18 and 24).

### EXAMPLE 29 OF THE INVENTION

Use of a Model Containing Mainly Interstitial Dendritic Cells in Suspension, Obtained According to Example 10 for Studying the Immunostimulant or Immunosuppressant Activity of an Active Principle or Evaluating and/or Inducing an Immunotolerance

The study is carried out according to the following protocol:
after stimulation with TNP, the cells are cultivated for 48 hours in culture medium possibly containing the active principles to be tested, at different concentrations; when the culture has ended, the phenotypic profile of the interstitial dendritic cells is studied by flow cytometry using the monoclonal antibodies anti-CD1a, anti-CCR6, anti-HLA-DR, anti-CD80, anti-CD83, anti-CD86, anti-CCR7 and anti-DC-LAMP.

The phenotypic profile of the cells makes it possible to define the immuno-modulating effect of the active principles tested.

### EXAMPLE 30 OF THE INVENTION

Use of Any One of the Models of Reconstructed Mucous Membrane Described in Example 21 or 22 for Studying Infection by HIV

The study is carried out according to the following protocol:

Infections are produced by the direct injection or deposition of the viral suspension (monocytotrophic strain HIV-1_{BaL} at a concentration of 55 nanograms p24/10⁶) in reconstructed mucous membranes after 35 days of culture using a needle. Incubation proceeds overnight at 37° C. and is followed by 4 washes with culture medium. The cultures are continued for one week and the following analyses are performed:
the viral replication is quantified by measuring the production of protein p24 in the culture supernatant of the infected reconstructed mucous membranes by ELISA (Coulter/Immunotech);
the infection of the DC is monitored by in situ PCR on histological sections of the infected reconstructed mucous membranes. The specific primers of the gag gene are SK38 and SK39, in the presence of digoxigenin labeled or unlabeled dNTP. The PCR conditions comprise a denaturation at 94° C. and 20 cycles at 95° C., 55° C. and 72° C. After the PCR, the sections are incubated with an antiDIG antibody couple with alkaline phosphatase. The sections are then stained with methyl green.
The results of in situ PCR show that there are infected cells in the epidermis (Langerhans cells) and in the dermis (interstitial dendritic cells).
This model can be used as a tool for studying the mechanisms of infection, replication and transmission of the virus and for researching and developing therapeutic methods (including vaccines, drugs etc.).

### EXAMPLE 31 OF THE INVENTION

Preparation of Suspensions of Dendritic Cells Using a Serum-Free Culture Medium—Therapeutic Applications

The CD14⁺ culture protocol is identical to Examples 2, 3, 4, 5, 6, 7 and 8. However, the RPMI 1640 medium supplemented with 10% of fetal calf serum is replaced with a specific serum-free medium from STEMBIO with the reference StembioA: SB A 100.

The dendritic cells can then serve as targets for sensitization and as therapeutic tools (antigen-presenting cells) in cell immunotherapy.

1. Use of CD14⁺ monocytes isolated from peripheral circulating blood for obtaining, by differentiation, at least one mixed population of Langerhans and interstitial dendritic cells, both Langerhans cells and interstitial dendritic cells being preconditioned and undifferentiated, and/or differentiated and immature, and/or mature, and/or interdigitated.

2. Use according to claim 1 wherein the differentiation results in the presence of at least one additional subpopulation of preconditioned and undifferentiated, and/or differentiated cells such as cells of the macrophage type and/or cells of the endothelial type.

3. Use according to claim 1 or 2 wherein the differentiation is effected by culture in a culture medium containing at least the two cytokines GM-CSF and TGFβ, preferably TGFβ₁.

4. Use according to claim 1, 2 or 3 wherein the distribution between the populations of Langerhans cells and interstitial dendritic cells depends on the presence of a third cytokine at a given concentration and for a given period of time during said culture, said cytokine preferably being the cytokine IL-13.

5. Use according to any one of the preceding claims wherein the culture is carried out in the presence of the cytokine IL-13 for at most about two days so as to favor differentiation into Langerhans cells.

6. Use according to any one of the claims 1 to 4 wherein the culture is carried out in the presence of the cytokine IL-13 for about six days in order to favor the formation of interstitial dendritic cells.

7. Use according to any one of the claims 1 to 4 wherein the culture is carried out in the presence of the cytokine IL-13 for about four days in order to favor the formation of a dual population of Langerhans cells/interstitial dendritic cells.

8. Use according to one of claims 1 to 7 wherein an additional degree of differentiation of Langerhans cells and interstitial dendritic cells is obtained by carrying out said culture in the presence of the cytokine TNFα.

9. Use according to claim 8 wherein the culture in the presence of TNα is carried out at a given concentration and for a given period of time, the latter being less than about 18 hours, in order to obtain the differentiation of still immature Langerhans cells and interstitial dendritic cells while at the same time avoiding a maturation into mature activated dendritic cells.

10. Use according to claim 8 wherein the culture in the presence of TNFα is carried out at a given concentration and for a given period of time, the latter being more than about 20 hours, in order to obtain a maturation into mature activated dendritic cells.

11. Use according to one of the preceding claims wherein the concentration of cytokine GM-CSF is between 0.1 and 4000 IU/ml and advantageously about 400 IU/ml, the concentration of cytokine TGFβ, preferably TGFβ₁, is between 0.01 and 400 ng/ml and advantageously about 10 ng/ml, the concentration of cytokine IL-13, if this cytokine is present in the medium, is between 0.01 and 400 ng/ml and advantageously about 10 ng/ml, and the concentration of cytokine TNFα, if this cytokine is present in the medium, is between 0.1 and 4000 IU/ml and advantageously about 200 IU/ml.

12. Use according to anyone of the preceding claims wherein, the extraction of CD14+monocytes is performed from fresh blood that is to say initiated and performed preferably not later than 24 hours after taking of blood on an individual, preferably not later than 18 hours, preferably not later than 12 hours, preferably not later than 6 hours and still preferably the extraction is immediately initiated just after the taking of blood and performed not later than 5 hours.

13. Use according to one of the preceding claims wherein the Langerhans cells and the interstitial dendritic cells generated have functional phenotypes identical to those found in vivo.

14. Use according to one of claims 1 to 13 wherein the culture of said Langerhans cells and interstitial dendritic cells is carried out in a three-dimensional culture environment comprising, in particular, at least epithelial and stromal cells.

15. Use according to one of the preceding claims wherein, when the epithelial and stromal cells are distinctly separated, the Langerhans cells are located preferentially in the region of the epithelial cells and the interstitial dendritc cells are located mainly in the region of the stromal cells.

16. Use according to any one of claims 1 to 15 wherein endothelial cells and macrophages are obtained by differentiation from certain cells derived from the culture, particularly when they are placed in a three-dimensional environment.

17. Use according to any one of claims 1 to 16 wherein cells, preferably preconditioned and undifferentiated cells, are obtained which, when integrated into a complete skin or mucous membrane model, i.e. a model comprising both an epithelium and a connective matrix, are capable, by virtue of the cellular environment, preferably fibroblasts and epithelial cells, and the matricial environment, of locating in the epithelium in order to differentiate into Langerhans cells, and in the connective matrix in order to differentiate into interstitial dendritic cells, macrophages and endothelial cells, and of acquiring a functionality comparable to that of Langerhans cells, interstitial dendritic cells, macrophages and endothelial cells in vivo.

18. Process for the in vitro culture of CD14⁺ monocytes which comprises:
a) the extraction, from peripheral circulating blood, of CD14⁺ monocytes previously harvested according to the state of the art, and
b) the culture of the separated CD14^{+monocytes in a culture medium containing several cytokines for a sufficient period of time to obtain a dual population of Langerhans cells and interstitial dendritic cells.}

19. Process according to claim 18 wherein the culture takes place in the presence of at least the cytokines GM-CSF and TGFβ, preferably TGFβ₁.

20. Process according to claim 18 or 19 wherein the culture takes place in the presence of a third cytokine at a given concentration and for a given period of time during said culture, said cytokine preferably being the cytokine IL-13.

21. Process according to one of claims 18 to 20 wherein the culture takes place in the presence of the cytokine IL-13 for at most about two days so as to favor differentiation into Langerhans cells.

22. Process according to one of claims 18 to 20 wherein the culture takes place in the presence of the cytokine IL-13 for about six days in order to favor the formation of interstitial dendritic cells.

23. Process according to one of claims 18 to 20 wherein the culture takes place in the presence of the cytokine IL-13 for about four days in order to favor the formation of a mixed population of Langerhans cells/interstitial dendritic cells.

24. Process according to one of claims 18 to 23 wherein the culture takes place in the presence of the cytokine TNFα.

25. Process according to claim 24 wherein the culture in the presence of TNFα is carried out at a given concentration and for a given period of time, the latter being less than about 18 hours, in order to obtain differentiation of the cells into Langerhans cells and still immature interstitial dendritic cells while at the same time avoiding a maturation into activated mature dendritic cells.

26. Process according to claim 24 wherein the culture in the presence of TNFα is carried out at a given concentration and for a given period of time, the latter being more than about 20 hours, in order to obtain a maturation into activated mature dendritic cells.

27. Process according to anyone of claims 18 to 26 wherein the extraction of CD14^{+monocytes is performed from fresh blood i.e initiated and performed preferably not later than} 24 hours after taking of blood on an individual, preferably not later than 18 hours, preferably not later than 12 hours, preferably not later than 6 hours and still preferably the extraction is immediately initiated just after the taking of blood and performed not later than 5 hours.

28. Process according to one of claims 18 to 27 wherein the culture takes place in the presence of a three-dimensional culture environment, particularly in the presence of at least epithelial cells and stromal cells.

29. Process according to one of claims 18 to 28 wherein an additional degree of differentiation is obtained by carrying out the culture of said Langerhans cells and interstitial dendritic cells in a three-dimensional culture environment comprising, in particular, at least distinctly separated epithelial and stromal cells.

30 Process according to one of claims 18 to 28 wherein, after culture with the cytokines, a complementary stimulation of maturation is effected in particular by interaction of the dendritic cells with CD40-ligand, or by addition of the cytokine TNFα or lipopolysaccharide, for a sufficient period of time to obtain a phenotypic and functional maturation of said cells.

31. Process according to one of claims 18 to 30 which comprises integration of at least a mixed population of Langerhans cells and interstitial dendritic cells, in variable proportions, into a three-dimensional culture model.

32. Process according to claim 31 wherein, the three-dimensional culture model includes skin models, mucous membrane models, dermis models, chorion models, epidermis models and epithelium models.

33. Process according to claim 31 or 32 wherein the three-dimensional culture model comprises a matricial support (of dermis or chorion) preferably selected from:
a collagen-based gel comprising stromal cells, particularly fibroblasts,
a porous matrix made of collagen which may contain one or more glycosaminoglycans and/or optionally chitosan, these porous matrices possibly integrating stromal cells, particularly fibroblasts,
a gel or a membrane of hyaluronic acid and/or of collagen and/or of fibronectin and/or fibrin,
a dermal equivalent constitued of dermal layers,
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester (HATF) membrane, a semipermeable Biopore-CM membrane and a semipermeable polyester membrane, a polyglycolic acid membrane or film, said inert support possibly containing stromal cells, particularly fibroblasts.

34. Process according to one of claims 31 to 33 wherein, the three-dimensional culture model used consists of the above-mentioned model onto whose surface epithelial cells, particularly keratinocytes, have been deposited.

35. Process according to one of claims 31 to 34 wherein, the three-dimensional culture model used consists of a model into which has been incorporated at least one complementary cell type, for example nerve cells and/or endothelial cells and/or melanocytes and/or lymphocytes and/or adipocytes and/or appendages of skin, such as scalp hair, other body hair and sebaceous glands.

36. Process according to one of claims 18 to 35 wherein, certain cells derived from the culture differentiate into endothelial cells and macrophages, particularly when they are placed in a three-dimensional environment comprising at least epithelial and stromal cells.

37. Culture process which comprises using CD14⁺monocytes in a manner described in any one of claims 1 to 17.

38. Medium for the in vitro culture of CD14+monocytes which comprises a basic culture medium combined with at least two cytokines, namely the cytokine GM-CSF and the cytokine TGFβ, preferably TGFβ₁.

39. Culture medium according to claim 38 wherein, the culture medium combined with said two cytokines is also combined with the cytokine IL-13, which is preferably physically separated so that it can be introduced into the culture medium at a given moment during culture.

40. Culture medium according to claim 38 or 39 wherein, the culture medium combined with said two cytokines is also combined with the cytokine TNFα, which is preferably physically separated so that it can be introduced into the culture medium at a given moment during culture.

41. Culture medium according to claims 38 to 40 wherein the concentration of cytokine GM-CSF is between 0.1 and 4000 IU/ml and advantageously about 400 IU/ml, the concentration of cytokine TGFα, preferably TGFβ₁, is between 0.01 and -400 ng/ml and advantageously about 10 ng/ml, the concentration of cytokine IL-13, if this cytokine is present in the medium, is between 0.01 and 400 ng/ml and advantageously about 10 ng/ml, and the concentration of cytokine TNFα, if this cytokine is present in the medium, is between 0.1 and 4000 IU/ml and advantageously about 200 IU/ml.

42. Cell populations comprising at least one mixed population of Langerhans cells and interstitial dendritic cells, both Langerhans cells and interstitial dendritic cells being preconditioned and undifferentiated and/or differentiated and immature and/or mature and/or interdigitated, which are obtainable from CD14⁺ monocytes and especially obtainable from CD14⁺monocytes as defined in any one of claims 1 to 17, or by the culture process according to any one of claims 18 to 37, or by the use of the culture medium as described according to any one of claims 38 to 41.

43. Use of at least the mixed population of Langerhans cells and interstitial dendritic cells obtained from the use of CD14+monocytes according to one of claims 1 to 17, or by a culture process according to one of claims 18 to 37, or by the use of the culture medium according to one of claims 38 to 41, or as defined in claim 42, for the manufacture of a suspension, monolayer or three-dimensional, monocellular or multicellular study model.

44. Use according to claim 43 wherein the study model comprises a support selected from:
a collagen-based gel comprising stromal cells, particularly fibroblasts,
a porous matrix made of collagen which may contain one or more glycosaminoglycans and/or optionally chitosan, these porous matrices possibly integrating stromal cells, particularly fibroblasts,
a gel or a membrane of hyaluronic acid and/or of collagen and/or of fibronectin and/or fibrin,
a dermal equivalent constitued of dermal layers,
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester (HATF) membrane, a semipermeable Biopore-CM membrane and a semipermeable polyester membrane, a polyglycolic acid membrane or film, said inert support possibly containing stromal cells, particularly fibroblasts.

45. Use according to claim 44 wherein, the study model comprises mainly either Langerhans cells, or interstitial dendritic cells, or a mixture of Langerhans cells/interstitial dendritic cells, or a mixture of Langerhans cells/interstitial dendritic cells/endothelial cells/macrophages, or a mixture of interstitial dendritic cells/endothelial cells/macrophages.

46. Complete model of reconstructed skin or reconstructed mucous membrane, or a model of reconstructed dermis or reconstructed chorion, or a model of reconstructed epithelium, particularly an epidermis model or any other suspension, monolayer or three-dimensional, monocellular or multicellular model, which comprises at least one mixed population as obtained in any one of claims 1 to 37 or as defined in claim 42.

47. Model according to claim 46 which comprises a support selected from:
a collagen-based gel comprising stromal cells, particularly fibroblasts,
a porous matrix made of collagen which may contain one or more glycosaminoglycans and/or optionally chitosan, these porous matrices possibly integrating stromal cells, particularly fibroblasts,
a gel or a membrane of hyaluronic acid and/or of collagen and/or of fibronectin and/or fibrin,
a dermal equivalent constitued of dermal layers,
a de-epidermized dead dermis,
an inert support selected from the group consisting of a semipermeable synthetic membrane, particularly a semipermeable nitrocellulose membrane, a semipermeable nylon membrane, a teflon membrane or sponge, a semipermeable polycarbonate or polyethylene or polypropylene or polyethylene terephthalate (PET) membrane, a semipermeable Anopore inorganic membrane, a cellulose acetate or ester.(HATF) membranes a semipermeable Biopore-CM membrane and a semipermeable polyester membrane, a polyglycolic acid membrane or film, said inert support possibly containing stromal cells, particularly fibroblasts.

48. Model according to claim 46 or 47 which comprises mainly either Langerhans cells, or interstitial dendritic cells, or a mixture of Langerhans cells/interstitial dendritic cells, or a mixture of Langerhans cells/interstitial dendritic cells/endothelial cells/macrophages, or a mixture of interstitial dendritic cells/endothelial cells/macrophages.

49. Model according to claims 46 to 48 wherein, the LC are located in the epithelial part and the IDC, macrophages and endothelial cells, when present, are located in the connective matrix.

50. Model according to one of claims 46 to 49 wherein, cells are present which provide architecture, especially stromal cells, particularly fibroblasts, and/or epithelial cells, particularly keratinocytes, and/or other cell types, especially T lymphocytes, and/or nerve cells, and/or pigmentary cells, particularly melanocytes, and/or adipocytes and cells which provide immune defense, especially Langerhans cells, interstitial dendritic cells and/or macrophages, and cells which provide vascularization, especially endothelial cells.

51. Use of at least the mixed population of Langerhans cells and interstitial dendritic cells as obtained according to one of claims 1 to 37 and 42 to 50 as a model for the study and/or selection of active principles.

52. Use of a model according to one of claims 43 to 50 especially for the purpose of studying the immunostimulant or immunosuppressant activity of an active principle or evaluating or inducing an immunotolerance by said active principle.

53. Use of a model according to one of claims 43 to 50 for studying the physiopathology of epithelial barriers; irritation of the skin or mucous membranes; aggressions of a biological nature, for example viruses, retroviruses such as HIV, bacteria, molds, microorganisms and particulate antigens; phototoxicity; photoprotection; the effect of active principles, particularly cosmetic or pharmaceutical active principles; and the effect of finished products, particularly cosmetic or pharmaceutical products; and for studying the mechanisms of infection by a pathogenic agent.

54. Use of a model according to one of claims 43 to 50 for detecting the presence of a pathogenic agent, for example viruses, retroviruses such as HIV, bacteria, molds, microorganisms and particulate antigens.

55. Use of a model described according to one of claims 43 to 50 for a medical, biomedical or cosmetic application, in particular for modulating the immune or tolerance response, in vitro or in vivo, following an environmental aggression, particularly of the physical type such as UV irradiation, or of the chemical or biological type, particularly for the purpose of preventive or curative therapy.

56. Use of a model described according to one of claims 43 to 50 for tissue and cell engineering applications.

57. Use of cell populations according to claim 42 for medical or biomedical applications such as anticancer cell therapy, for example an injection of DC capable of stimulating the immune response; cell therapy in cases of autoimmune disease through the creation of an immunotolerance situation, for example by producing anergic T cells; gene therapy for diseases affecting the immune system; and the development and production of vaccines.
